SEQUENCE LISTING (4 sequences)

SEQ ID NO: 1 - DNA, 37 bp, Artificial Sequence
  forward primer for nucleotides 151 to 1800 of Staphylococcus aureus SdrD A gene
cgcaggatcccaggcagaaagtactaataaagaattg

SEQ ID NO: 2 - DNA, 34 bp, Artificial Sequence
  reverse primer for nucleotides 151 to 1800 of Staphylococcus aureus SdrD A gene
cgcagtcgacttcttgaccagctccgccactttg

SEQ ID NO: 3 - protein, 516 aa, Artificial Sequence
  amino acids 53-569 of recombinant SdrD A-region protein in Staphylococcus aureus
ESTNKELNEATTSASDNQSSDKVDMQQLNQEDNTKNDNQKEMVSSQGNETTSNGNKLIEKESVQSTTGNKVEVSTAKSDEQASPKSTNEDLNTKQTISNQEALQPDLQENKSVVNVQPTNEENKKVDAKTESTTLNVKSDAIKSNDETLVDNNSNSNNENNADIILPKSTAPKRLNTRMRIAAVQPSSTEAKNVNDLITSNTTLTVVDADKNNKIVPAQDYLSLKSQITVDDKVKSGDYFTIKYSDTVQVYGLNPEDIKNIGDIKDPNNGETIATAKHDTANNLITYTFTDYVDRFNSVQMGINYSIYMDADTIPVSKNDVEFNVTIGNTTTKTTANIQYPDYVVNEKNSIGSAFTETVSHVGNKENPGYYKQTIYVNPSENSLTNAKLKVQAYHSSYPNNIGQINKDVTDIKIYQVPKGYTLNKGYDVNTKELTDVTNQYLQKITYGDNNSAVIDFGNADSAYVVMVNTKFQYTNSESPTLVQMATLSSTGNKSVSTGNALGFTNNQSGGAGQEV

SEQ ID NO: 4 - protein, 336 aa, Artificial Sequence
  amino acids 234-569 of N2N3 domain of recombinant SdrD A-region protein in Staphylococcus aureus
IAAVQPSSTEAKNVNDLITSNTTLTVVDADKNNKIVPAQDYLSLKSQITVDDKVKSGDYFTIKYSDTVQVYGLNPEDIKNIGDIKDPNNGETIATAKHDTANNLITYTFTDYVDRFNSVQMGINYSIYMDADTIPVSKNDVEFNVTIGNTTTKTTANIQYPDYVVNEKNSIGSAFTETVSHVGNKENPGYYKQTIYVNPSENSLTNAKLKVQAYHSSYPNNIGQINKDVTDIKIYQVPKGYTLNKGYDVNTKELTDVTNQYLQKITYGDNNSAVIDFGNADSAYVVMVNTKFQYTNSESPTLVQMATLSSTGNKSVSTGNALGFTNNQSGGAGQEV

(19) United States
(12) Patent Application Publication
(10) Pub. No.: US 2009/0162379 A1
(43) Pub. Date: Jun. 25, 2009

(54) Inhibitors of S. aureus SdrD protein attachment to cells and uses therefor

(76) Inventors: Magnus Hook, Houston, TX (US); Elena M. Barbu, Houston, TX (US)

Correspondence Address: Benjamin Aaron Adler;ADLER & ASSOCIATES, 8011 Candle Lane, Houston, TX, 77071, US

(21) Appl. No.: 12/286,586

(22) Filed: Oct. 1, 2008

Related U.S. Application Data
(60) Provisional application No. 60/997,129, filed on Oct. 1, 2007.

(51) Int. Cl. A61K 39/40; G01N 33/569; G01N 33/53; C07K 14/31; A61K 38/16; C12N 5/00; C12N 1/20; C07K 16/00; A61P 31/04

(52) U.S. Cl. 424/165.1; 435/7.33; 435/7.21; 530/350; 514/12; 435/375; 435/252.1; 530/389.5

(57) ABSTRACT

Provided herein is a method for identifying small molecule inhibitors of S. aureus SdrD protein attachment to a host cell receptor using a structural model of SdrD protein-receptor interaction. Also provided are the small molecule inhibitors so identified and synthetic small molecules effective to bind SdrD protein and/or the host cell receptor. In addition, antibodies directed against SdrD protein are provided. Further provided are methods of treating or preventing S. aureus associated lung infections and of inhibiting S. aureus adherence to a lung cell using the small molecules and antibodies described herein.

### CROSS-REFERENCE TO RELATED APPLICATIONS

This non-provisional application claims benefit of provisional application U.S. Ser. No. 60/997,129 filed on Oct. 1, 2007, now abandoned.

### FEDERAL FUNDING LEGEND

This invention was produced in part using funds obtained through grant A120624 from the National Institutes of Health. Consequently, the federal government has certain rights in this invention.

### BACKGROUND OF THE INVENTION

1. Field of the Invention

The present invention relates to the fields of pathogenic microbiology, drug design and medicine. Specifically, the present invention relates to inhibitors of S. aureus SdrD protein attachment to a host cell and methods for treating or preventing S. aureus infections and other conditions using the inhibitors.

2. Description of the Related Art

S. aureus is a versatile and frequent human pathogen in both community and hospitals settings. Diseases caused by this organism range from mild skin infections to life-threatening pneumonia, endocarditis and sepsis. Being primarily an extracellular pathogen, its ability to initiate infection resides in adherence to the extracellular matrix of the host cells or directly to exposed cells to initiate colonization. Proteins involved in adherence are mostly cell wall anchored and belong to a family of structurally related proteins named microbial surface components recognizing adhesive matrix molecules (MSCRAMM).

S. aureus causes infections due to its ability to adhere to components of the extracellular matrix of the host. Bacteria initiate colonization through binding to fibronectin via FnbpA, FnbpB, fibrinogen, i.e., ClfA, ClfB and FnbpA, and collagen (Cna). Some microbial surface components recognizing adhesive matrix molecules, particularly SdrD, are overexpressed in S. aureus during lung infections [1] such that S. aureus show an increased tissue adherence and colonization [2].

Currently, the standard treatment for pneumonia and other diseases caused by S. aureus is antibiotic based therapy. Increasing resistance, high rates of complications and relapsing infections indicate that current therapies are insufficient. Immunization therapies have been examined. Animal studies showed that administration of monoclonal antibodies was protective in mice against sepsis-induced mortality [3]. Also, monoclonal antibody therapy enhanced the efficacy of the antibiotic treatment in a rabbit model of infective endocarditis by reducing levels of bacteria in blood, vegetations and organs [4]. In addition a phase II trial demonstrated that progression of sepsis was more pronounced in the placebo group compared with the antibody treated group [5]. Small molecule inhibitor therapy has been proposed as treatment for infection with Pseudomanas aeruginosa [6], HIV [7], hepatitis C virus [8] and other infectious agents. However, no candidate is available for S. aureus infection.

Thus, a recognized need is still present in the art for disruptors of SdrD-receptor interaction effective to decrease attachment of bacteria to lung epithelium thereby promoting increased bacterial clearance. Specifically, the prior art is deficient in small molecule inhibitors of S. aureus SdrD protein effective to treat or prevent pathophysiological conditions associated with S. aureus infection of the lung. The present invention fulfills this long standing need in the art.

### SUMMARY OF THE INVENTION

The present invention is directed to a method for identifying a small molecule inhibitor of S. aureus (S. aureus) SdrD protein attachment to a host cell receptor. The method comprises designing a test compound that binds to one or both of an SdrD protein or fragment thereof or SdrD receptor based on a structural model of SdrD protein-receptor interaction and measuring adherence of an S. aureus bacteria overexpressing SdrD to a host cell comprising the receptor in the presence and in the absence of the test compound. The level of S. aureus adherence is compared in the presence of the test compound with the level of S. aureus adherence in the absence of the test compound, where a decrease in adherence in the presence of the test compound is indicative that the test compound is a small molecule inhibitor of SdrD protein attachment to the host cell receptor. The present invention is further directed to a related method comprising an additional step of screening the small molecule inhibitor for cytotoxicity to the host cell. The present invention is directed to another related method comprising an additional step of determining a therapeutic index of the small molecule inhibitor.

The present invention also is directed to a small molecule inhibitor identified by the method described herein. The present invention is directed to a related synthetic small molecule effective to bind to S. aureus SdrR protein. The presented invention also is directed to another related synthetic small molecule effective to bind to an S. aureus protein receptor.

The present invention is directed further to a method for treating or preventing a S. aureus infection of the lung in a subject in need thereof. The method comprises administering to the subject a pharmacologically effective amount of one or more of the small molecule inhibitors described herein.

The present invention is directed further still to a method for inhibiting adherence of S. aureus bacteria to a lung cell. The method comprises contacting one or both of the S. aureus bacteria overexpessing SdrD protein or the lung cell with an amount of one or more of the inhibitors described herein effective to interfere with attachment of S. aureus SdrD protein to its receptor on the lung cell thereby inhibiting adherence of the S. aureus bacteria to the lung cell.

The present invention is directed further still to an antibody directed against S. aureus SdrD protein.

The present invention is directed further still to a method for treating or preventing an S. aureus-associated pneumonia in a subject. The method comprises administering to the subject the antibody described herein.

Other and further aspects, features and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions and certain embodiments of the invention briefly summarized above are illustrated in the appended drawings. These drawings form a part of the specification. It is to be noted, however, that the appended drawings illustrate preferred embodiments of the invention and therefore are not to be considered limiting in their scope.

FIG. 1 demonstrates adherence of different S. aureus strains to A549 cells.

FIG. 2 demonstrates attachment to A549 cells.

FIG. 3A demonstrates the interaction between SdrD⁺ L. lactis (bright red) and A549 cells (pale red due to autofluorescence) using fluorescence microscopy. L. lactis cells are labeled with Texas Red.

FIG. 3B is the differential contrast image of A549 cells.

FIG. 4 demonstrates inhibition of bacterial attachment to A549 by polyclonal antibodies specifically recognizing SdrD.

FIGS. 5A-5B shows that recombinant SdrD A-region inhibits bacterial colonization mediated by SdrD. FIG. 5A shows A549 cells were incubated overnight with 40 μM recombinant protein (SdrD A or ClfB A) or FIG. 5B, A549 cells were incubated overnight with 0 to 100 μM recombinant SdrD A before the attachment assay was performed. The adherence assay was carried out for 45 minutes at 37° C. in humidified chamber with 5% CO₂. Data presented represent the mean±SD of 2 independent experiments performed in triplicate. p value was calculated using the Student's t-test.

FIGS. 6A-6B show that anti-SdrD antibodies inhibit SdrD-mediated bacterial colonization. FIG. 6A shows that bacteria were incubated 2 hours at room temperature with 50 μg antibodies/ml (anti-SdrD or anti-ClfB). FIG. 6B shows that bacteria were incubated with 0 to 60 μg antibodies/ml before the attachment assay was performed. Data presented represent the mean±SD of two independent experiments performed in triplicate. p value was calculated using the Student's t-test.

FIGS. 7A-7B show the amino acid sequence SEQ ID NO: 3 of recombinant SdrD-A region protein (FIG. 7A) and the amino acid sequence SEQ ID NO: 4 of the N2N3 domain within SdrD-A region protein (FIG. 7B).

### DETAILED DESCRIPTION OF THE INVENTION

As used herein, the term “a” or “an”, when used in conjunction with the term “comprising” in the claims and/or the specification, may refer to “one”, but it is also consistent with the meaning of “one or more”, “at least one”, and “one or more than one”. Some embodiments of the invention may consist of or consist essentially of one or more elements, method steps, and/or methods of the invention. It is contemplated that any compound, composition, or method described herein can be implemented with respect to any other device, compound, composition, or method described herein.

As used herein, the term “or” in the claims refers to “and/or” unless explicitly indicated to refer to alternatives only or the alternatives are mutually exclusive, although the disclosure supports a definition that refers to only alternatives and “and/or”.

As used herein, the term “small molecule inhibitor” is interchangeable with “inhibitor”, or “inhibitory compound” and means a molecular entity of natural, semi-synthetic or synthetic origin that blocks, stops, inhibits, and/or suppresses S. aureus SdrD protein interactions with a host cell SdrD ligand or receptor.

As used herein, the term “contacting” refers to any suitable method of bringing one or more of the small molecule compounds described herein or other inhibitory agent into contact with an S. aureus SdrD protein and/or its host cell ligand or receptor, as described, or a cell comprising the same. In vitro or ex vivo this is achieved by exposing S. aureus and/or host cells to the small molecule inhibitor or inhibitory agent in a suitable medium. For in vivo applications, any known method of administration is suitable as described herein.

As used herein, the terms “effective amount” or “pharmacologically effective amount” are interchangeable and refer to an amount that results in an improvement or remediation of the symptoms of an S. aureus-associated disease, disorder or condition, preferably of the lung, more preferably pneumonia. Those of skill in the art understand that the effective amount may improve the patient's or subject's condition, but may not be a complete cure of the disease, disorder and/or condition.

As used herein, the term “inhibit” refers to the ability of the small molecule to block, partially block, interfere, decrease, reduce S. aureus SdrD attachment to a host cell or extracellular membrane thereof. Thus, one of skill in the art understands that the term inhibit encompasses a complete and/or partial loss of attachment to its ligand or receptor. S. aureus SdrD attachment may be inhibited by disruption of SdrD interaction with the host cell ligand or receptor, by binding to the SdrD protein and/or to the host cell ligand or receptor, or by other means. For example, a complete and/or partial inhibition of SdrD attachment may be indicated by an increase in S. aureus clearance from the body.

As used herein, the term “treating” or the phrase “treating an S. aureus infection” or “treating S. aureus-associated pneumonia” includes, but is not limited to, halting the attachment of S. aureus to a host cell or extracellular membrane thereof. Treating an S. aureus infection, particularly a lung infection, encompasses therapeutic administration of the small molecule inhibitor(s) described herein singly or in combination with other known therapeutic agents or pharmaceuticals.

As used herein, the term “subject” refers to any recipient of a small molecule inhibitor effective to inhibit S. aureus attachment to a host cell or extracellular membrane thereof as a prophylactic or treatment for an S. aureus-infection of the lung.

In one embodiment of the present invention, there is provided a method for identifying a small molecule inhibitor of S. aureus (S. aureus) SdrD protein attachment to a host cell receptor, comprising designing a test compound that binds to one or both of an SdrD protein or fragment thereof, such as Region A or N2N3, or SdrD receptor based on a structural model of SdrD protein-receptor interaction; measuring adherence of an S. aureus bacteria overexpressing SdrD to a host cell comprising the receptor in the presence and in the absence of the test compound; and comparing the level of S. aureus adherence in the presence of the test compound with the level of S. aureus adherence in the absence of the test compound, wherein a decrease in adherence in the presence of the test compound is indicative that the test compound is a small molecule inhibitor of SdrD protein attachment to the host cell receptor.

Further to this embodiment the method comprises screening the small molecule inhibitor for cytotoxicity to the host cell. In another further embodiment, the method comprises determining a therapeutic index of the small molecule inhibitor. In all embodiments, a representative host cell may be a lung epithelial cell. In another embodiment of the present invention there is provided a small molecule inhibitor identified by method as described supra.

In yet another embodiment of the present invention there is provided an inhibitor of S. aureus SdrD protein attachment to a host cell comprising a recombinant SdrD A-region having the sequence shown in SEQ ID No. 3 or SEQ ID No. 4. A person having ordinary skill in this art could readily manipulate the sequences shown in SEQ ID No. 3 or SEQ ID No. 4 to develop a similar inhibitor that is not 100% identical to either sequence shown in SEQ ID No. 3 or SEQ ID No. 4. Accordingly, the present invention also encompasses an inhibitor having at least 95% homology to SEQ ID No. 3 or SEQ ID No. 4, an inhibitor has at least 90% homology to SEQ ID No. 3 or SEQ ID No. 4 or an inhibitor has at least 80% homology to SEQ ID No. 3 or SEQ ID No. 4. Further, the present invention also provides for a pharmaceutical composition comprising the inhibitor described herein.

In yet another embodiment of the present invention there is provided a method for treating or preventing a S. aureus infection of the lung in a subject in need thereof, comprising: administering to the subject a pharmacologically effective amount of one or more of the inhibitors described herein. This method may further comprise administering one or more other therapeutic agents effective to treat the lung infection which may be administered concurrently or sequentially with the inhibitor. In one aspect, this method may be used to treat is a nosocomial infection or pneumonia.

In yet another embodiment of the present invention there is provided a method for inhibiting adherence of S. aureus bacteria to a lung cell, comprising: contacting one or both of an S. aureus bacteria overexpessing SdrD protein or the lung cell with an amount of one or more of the inhibitors effective to interfere with attachment of S. aureus SdrD protein to its receptor on the lung cell thereby inhibiting adherence of the S. aureus bacteria. The present invention also encompasses a synthetic small molecule effective to bind to S. aureus SdrD protein or protein receptor.

In yet another embodiment of the present invention there is provided an antibody directed against S. aureus SdrD protein. In one aspect the SdrD protein is region A of the SdrD protein. In another aspect, the SdrD protein is the N2N3 region of the SdrD protein.

In yet another embodiment of the present invention there is provided a method for treating or preventing an S. aureus-associated infection in a subject, comprising administering to the subject an antibody described herein.

The present invention discloses, inter alia, that (1) SdrD binds to a protein ligand on mammalian (including human) cells; (2) SdrD is a key adhesin mediating staphylococcal adherence to mammalian (particularly lung epithelial) cells; and (3) the A region (and more specifically the N2N3 domains) contain the binding site for the mammalian cell ligand. Thus, a person having ordinary skill in this art would readily recognize that one may use this finding to design a screening method for identifying an inhibitor of mammalian cells with SdrD and design a screening method for the development of a therapeutic/preventive monoclonal antibody that inhibits SdrD mammalian cell interactions. This monoclonal antibody should be directed to the A region and more preferably the N2N3 domains. In addition, SdrD is a viable vaccine component in a staphylococcal vaccine. The SdrD vaccine component includes the A region and more preferably the N2N3 domain.

It is demonstrated herein that SdrD is an adhesin belonging to the MSCRAMM protein family and contributes to the attachment of S. aureus to lung epithelial cells. Overexpression of SdrD may enhance tissue adherence and promote colonization during an S. aureus lung infection. It is contemplated that inhibition of the interaction between SdrD and its receptor will increase the clearance of bacteria from lungs.

Thus, small molecule inhibitors of S. aureus SdrD protein attachment to lung epithelial cells are provided. A structural model of SdrD protein-ligand interaction is used to design or screen test compounds as potential small molecule inhibitors of SdrD attachment to its host receptor. Potential inhibitory compounds may be designed de nova, including computer-aided design. De nova compounds may be synthesized by known chemical synthetic routes. Alternatively, libraries of known small molecules may be screened as inhibitors of the interaction between SdrD and its receptor. Efficacy of the identified small molecule inhibitors may be determined using adherence assays known and standard in the art. In addition the therapeutic index or any cytotoxic effects of the inhibitors on the host cell of the identified inhibitors may be determined by standard methods known to those skilled in the art.

Thus, the small molecule inhibitors provided herein are useful as therapeutics. The inhibitory compounds provided herein may be used to treat any subject, preferably a mammal, more preferably a human having an S. aureus-associated lung infection, such as, but not limited pneumonia. The lung infection may be a nosocomial infection. It is contemplated that contacting the S. aureus bacteria with one or more of these small molecule inhibitors is effective to at least inhibit, reduce or prevent S. aureus SdrD attachment to lung cells or the extracellular matrix thereof. The small molecule inhibitors of the present invention may be administered alone or in combination or in concurrent therapy with other therapeutic agents or pharmaceuticals which affect the lung infection or other concurrent pathophysiological condition.

The present invention also contemplates therapeutic methods employing compositions comprising the small molecule inhibitors disclosed herein. Preferably, these compositions include pharmaceutical compositions comprising a therapeutically effective amount of one or more of the small molecule inhibitors along with a pharmaceutically acceptable carrier. Also, these pharmacological compositions may include pharmacologically effective salts or hydrates of the inhibitors.

As is well known in the art, a specific dose level of active compounds, such as SdrD small molecule inhibitors or related- derivative or analog compounds thereof for any particular patient depends upon a variety of factors including the activity of the specific compound employed, the age, body weight, general health, sex, diet, time of administration, route of administration, rate of excretion, drug combination, and the severity of the S. aureus infection undergoing therapy. The person responsible for administration is well able to determine the appropriate dose for the individual subject and whether a suitable dosage of either or both of the small molecule inhibitor(s) and other therapeutic agent(s) comprises a single administered dose or multiple administered doses.

It is also contemplated that antibodies specifically recognizing SdrD protein are useful as therapeutic agents in the treatment or prevention of an S. aureus-associated infection, particularly lung infection, such as, but not limited to, pneumonia. Methods of generating and characterizing antibodies are well-established in the molecular biological arts. Anti-SdrD antibodies may comprise an immunogenic composition, including an immunologically acceptable adjuvant or diluent. Therapeutic efficacy of anti-SdrD antibodies may be tested in, for example, a pneumonia animal model.

The present invention is also directed to an immunogenic composition that comprises a protein from SdrD A-region having the sequence shown in SEQ ID No. 3 or SEQ ID No. 4. Preferably, the immunogenic composition contains a protein that has at least 95% homology to SEQ ID No. 3 or SEQ ID No. 4. Even more preferably, the immunogenic composition contains a protein that has at least 90% or 80% homology to SEQ ID No. 3 or SEQ ID No. 4. Furthermore, as would be well known to those having ordinary skill in this art, the immunogenic composition may further comprise an adjuvant or diluent.

The following example(s) are given for the purpose of illustrating various embodiments of the invention and are not meant to limit the present invention in any fashion.

### EXAMPLE1

Adherence of S. aureus to A549 Cells

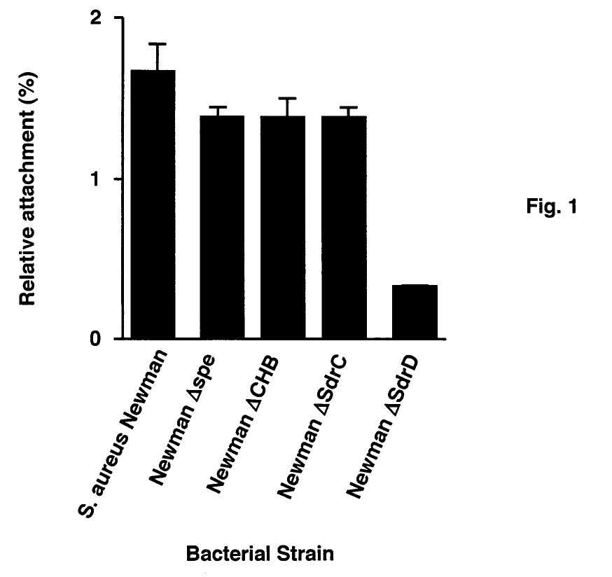

The lung alveolar epithelial cell line, A549, is used to demonstrate that S. aureus strains overexpressing SdrD exhibit increased adherence to the epithelium whereas an S. aureus Newman strain deficient in SdrD has decreased adherence (FIG. 1). S. aureus strains were grown to exponential phase and adherence to A549 cess was measured using a standard adherence assay method. The relative attachment was calculated as a percent of bacterial cells added. S. aureus Newman is a wild-type strain. The other strains are different isogenic microbial surface components recognizing adhesive matrix molecule mutants.

### EXAMPLE 2

L. lactis Attachment to A549 Cells

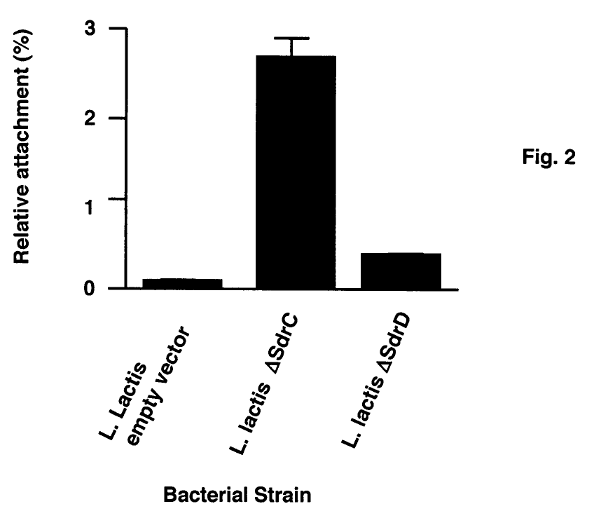

SdrD was expressed in a heterologous system, L. lactis, which does not possess any known adhesins. Expression of SdrD in L. lactis (L. lactis SdrD⁺) resulted in high attachment to alveolar epithelial cells in comparison with L. lactis cells bearing an empty vector (FIG. 2). Bacterial strains were grown to stationary phase when expression of microbial surface components recognizing adhesive matrix molecules is maximal. The aderence to A549 cells was measured using a standard adherence assay. Relative attachment was calculated as a percent of total bacterial cells added.

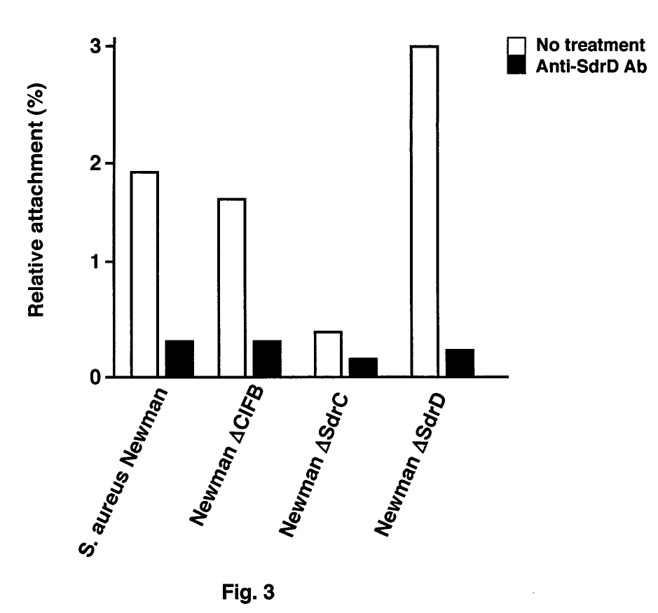

Fluorescence microscopy demonstrated that S. aureus and L. lactis SdrD+ attach to A549 cells (FIGS. 3A-3B). L. lactis SdrD+ cells (bright red), recognized by a rabbit anti-SdrD antibody and a goat anti-rabbit Texas Red antibody, are visualized attached to A549 cells (light red due to autofluorescence). L. lactis SdrD+ cells were incubated with A549 cells for 1 hour, fixed with 2.5% p-formaldehyde and detected with a rabbit polyclonal antibody specifically recognizing SdrD. A Texas Red goat anti-rabbit antibody is used as a secondary antibody.

### EXAMPLE 3

### Anti-SdrD Polyclonal Antibody Inhibition of SdrD Attachment to A549

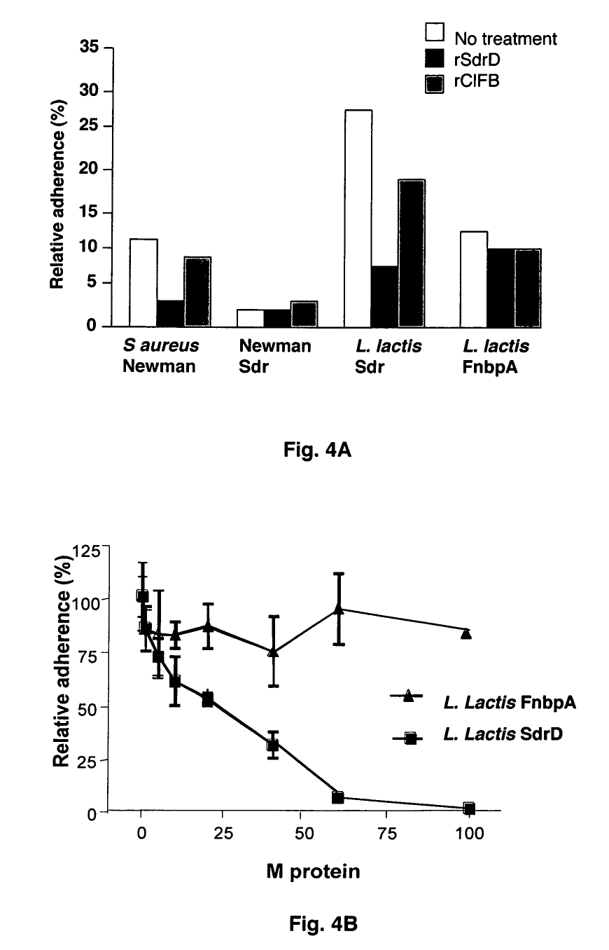

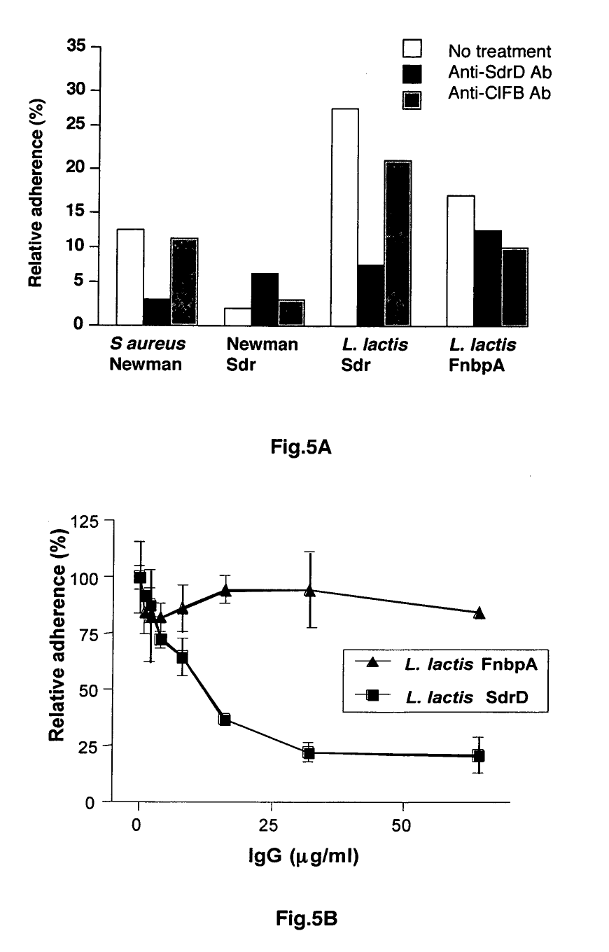

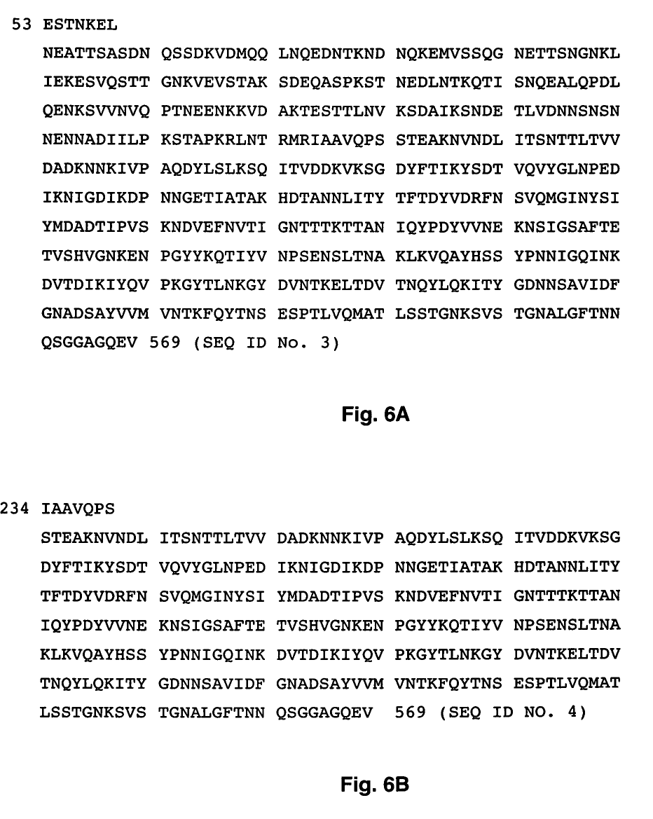

Purified polyclonal antibodies specifically recognizing SdrD inhibit adherence of S. aureus and L. lactis SdrD+ to A549 cells. An antibody recognizing another microbial surface components recognizing adhesive matrix molecule, i.e., ClfB, had no effect on bacterial attachment to lung epithelium (FIG. 4). Bacterial strains were grown to exponential phase and incubated for 1 hr with rabbit antid-SdrD antibodies. The attachment was measured using a standard bacterial adherence assay. Results were reported as percent of total bacteria added.

### EXAMPLE 4

The present invention focused on the identification of inhibitors of SdrD-mediated staphylococcal adherence to host cells. To achieve this goal, the presence of the SdrD receptor on lung epithelium was identified and the role of SdrD in a pneumonia murine model was established and the protection conferred by the anti-SdrD sera was shown.

### SdrD Receptor is Present on the Surface of Lung Epithelial Cells

To investigate the nature and localization of the SdrD receptor on the alveolar epithelial cells, SdrD-mediated bacterial adherence to A549 cells was assessed using fluorescence microscopy. A549 cells were grown on glass coverslips to 70% confluence and then infected with SdrD⁺L. lactis. After the removal of unbound bacteria, eukaryotic cells were stained with SP-DIOC₁₈, a lipophilic dye which inserts in the membrane, and then fixed in paraformaldehyde.

To visualize attached bacteria, coverslips were with a polyclonal anti-SdrD antibody followed by a Texas Red-labeled goat anti-rabbit antibody. This experiment served two purposes: first to confirm that bacteria expressing SdrD attach to A549 cells and second to determine if bacterial cells adhere to the surface of the epithelial cells or to the basolateral laid extracelullar matrix. The data showed that bacterial cells always co-localize with A549 cells, indicating that SdrD receptor is most likely a protein associated with the surface of the epithelial cell or a transmembrane protein.

The A549 cells in a 70% confluent monolayer are not yet differentiated, and therefore do not exhibit a basolateral and an apical surface. To simulate the in vivo conditions polarized A549 cells were also used. The adherence of SdrD⁺ L. lactis to basal ECM using a modified adherence assay was further examined. Confluent monolayer or polarized A549 cells were grown in 24 well plates and then cells were lifted by lysis with sterile water prior bacterial addition. The removal of A549 cells was confirmed by bright field microscopy and the immobilization of the basal matrix proteins was detected using bicinchronic acid assay. As expected, adherence of L. lactis SdrD to ECM proteins was comparable to their attachment to uncoated plastic suggesting that the SdrD receptor is not a basal matrix component.

### SdrD Receptor is a Protein

To determine the nature of the receptor, monolayer and polarized A549 cells were treated with periodic acid, lipase, proteinase K or trypsin, prior to infection to destroy carbohydrates, lipids or proteins respectively without destroying the layer of cells. The data showed that the attachment of SdrD⁺ L. lactis was significantly reduced only when epithelial cells were treated with proteinase K or trypsin. Taken together, these results indicate that the SdrD receptor is a protein located on the apical surface of lung epithelial cells.

### SdrD Promotes Adherence to Airway Epithelium in Vivo

To demonstrate the ability of SdrD to mediate adherence of bacteria to the airway epithelium, adult BALB/c mice were infected intranasally with L. lactis bearing an empty vector or L. lactis expressing SdrD. Three hours after infection, the lungs were lavaged with PBS to remove unbound bacteria and collected the lavage fluid.

The data showed that a significantly higher number of bacteria were recovered from lungs (p<0.01) when animals were infected with L. lactis SdrD, whereas approximately the same number of bacteria were found in the bronchoalveolar lavage fluids from mice infected with either strain. These data support the role of SdrD in attachment to healthy airway epithelia in vivo.

### EXAMPLE 5

### Recombinant SdrD A-region Purification

For plasmid construction, DNA manipulation was performed using standard methods. DNA modification and restriction enzymes were purchased from New England Biolabs, Inc. or Promega and used according to the manufacturer's instructions. The gene fragment encoding SdrD A (nucleotides 151 to 1800) was amplified by polymerase chain reaction from S. aureus Newman genomic DNA using the oligonucleotides forward 5′CGCAGGATCCCAGGCAGMAGTACTAATAAAGAATTG (SEQ ID No. 1) and reverse 5′ CGCAGTCGACTTCTTGACCAGCTCCGCCACTTTG (SEQ ID No. 2). The PCR product was analyzed by agarose gel electrophoresis, purified using QlAquick the gel extraction kit (Qiagen Sciences, Maryland) and cloned into pQE30 (Qiagen Sciences, Maryland). The plasmid was sequenced to ensure the integrity of the amplified fragments (Baylor DNA Sequencing Core Facility).

For the expression and purification of recombinant proteins, recombinant plasmids pQE30-SdrD A, pQE30-ClfB A, SpAD-GST, SpADF13A-GST or SpADY14A-GST were transformed into E. coli TOPP 3 (Stratagene, La Jolla, Calif.). Overnight starter cultures were diluted 1:50 in LB containing ampicillin (100μg/ml) and incubated with shaking at room temperature or 37° C. until the culture reached OD₆₀₀ 0.6-0.8. Protein expression was induced by addition of 0.1 mM IPTG; cells were incubated with shaking for additional 4 hours. Bacterial cells were harvested by centrifugation, resuspended in PBS containing EDTA-free Complete Protease Inhibitor (Roche Diagnostics, Mannheim, Germany) and frozen at −80° C.

Cells containing recombinant protein fragments were passed through a French press (1100 p.s.i.). Cellular debris was removed by centrifugation at 100,000× g for 20 minutes and filtration through a 0.45 μM membrane. To purify SdrD A and ClfB A, filtered cell lysate was applied at 2 ml/min to a 5 ml nickel-charged HiTrap Chelating column (GE Healthcare, Uppsala, Sweden) equilibrated with 10 mM Tris HCl, 100 mM NaCl pH 7.9. The column was washed with 40 volumes of 10 mM Tris HCl, 100 mM NaCl, 20 mM imidazole. Bound protein was eluted with a linear gradient of imidazole (10 to 200 mM, total volume 200 ml). This purification step yielded proteins that were more than 95% pure. Fractions containing recombinant protein were dialyzed in overnight in 4 L of 25 mM Tris-Cl pH 7.9 containing 10 mM EDTA and 1 mM 1.10 O-phenantroline. Sample was applied at 2 ml/minute to a HiTrapQ anion exchange column (GE Healthcare, Uppsala, Sweden) equilibrated with 25 mM Tris-Cl pH 7.9, to remove contaminating proteases. The recombinant protein of interest was collected from the flow through and dialyzed against PBS pH 7.4. Dialyzed sample was reapplied on a nickel-charged HiTrap Chelating column, and reapplied to a 5 ml nickel-charged HiTrap Chelating column. Pure protein was dialyzed against PBS, and applied to an Endotoxi-Gel Column (Pierce, Rockland, Ill.) to remove traces of LPS. Proteins used in these studies had less than 10 pg/ml LPS according to the Limulus Amebocyte Lysate Assay (Fisher Scientific, Suwannee, Ga.).

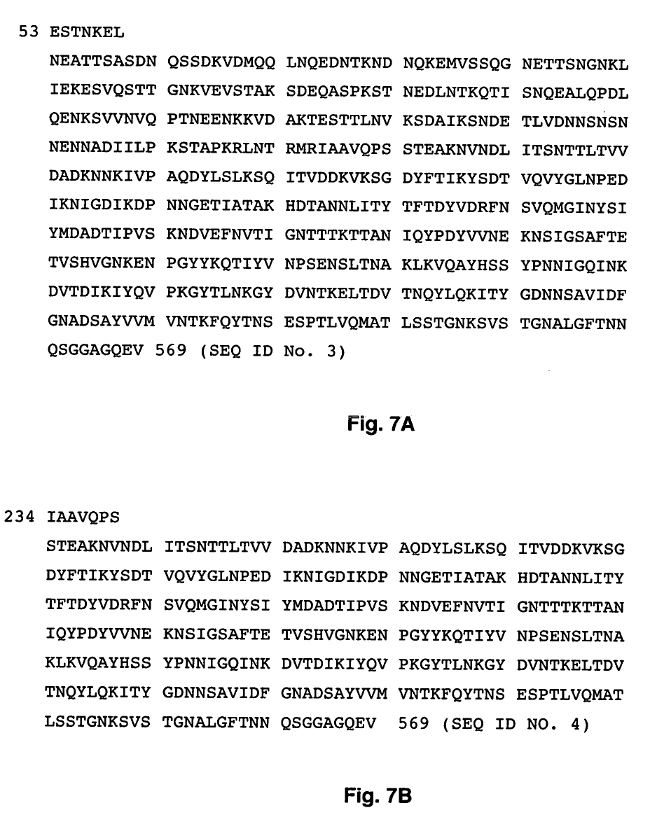

FIGS. 7A-7B show the amino acid sequence of recombinant SdrD A-region protein (aa 53-569; SEQ ID NO: 3) from S. aureus and of the recombinant N2N3 domain (aa 234-569; SEQ ID NO: 4) therein.

The following references are cited herein.
1. Labandeira-Rey et al. (2007) Science, 315(5815):1130-1133.
2. de Bentzmann et al. (2004) J Infect Dis, 190(8):1506-1515.
3. Hall et al. (2003) Infect Immun, 71(12):6864-6870.
4. Domanski et al. (2005) Infect Immun, 73(8):5229-5232.
5. Weems et al. (2006) Antimicrob Agents Chemother, 50(8):2751-2755.
6. Geske et al. (2005) J Am Chem Soc, 127(37):12762-12763.
7. Debnath, AK (2006) Expert Opin Investig Drugs, 15(5):465-478.
8. Del Vecchio and Sarisky (2006) Mini Rev Med Chem, 6(11):1263-1268.

Any patents or publications mentioned in this specification are indicative of the levels of those skilled in the art to which the invention pertains. These patents and publications are incorporated by reference herein to the same extent as if each individual publication was incorporated by reference specifically and individually. One skilled in the art will appreciate that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

What is claimed is:

1. A method for identifying a small molecule inhibitor of S. aureus (S. aureus) SdrD protein attachment to a host cell receptor, comprising:
designing a test compound that binds to one or both of an SdrD protein or fragments thereof or SdrD receptor based on a structural model of SdrD protein-receptor interaction;
measuring adherence of an S. aureus bacteria overexpressing SdrD to a host cell comprising the receptor in the presence and in the absence of the test compound; and
comparing the level of S. aureus adherence in the presence of the test compound with the level of S. aureus adherence in the absence of the test compound, wherein a decrease in adherence in the presence of the test compound is indicative that the test compound is a small molecule inhibitor of SdrD protein attachment to the host cell receptor.

2. The method of claim 1, further comprising:
screening the small molecule inhibitor for cytotoxicity to the host cell.

3. The method of claim 1, wherein the host cell is a lung epithelial cell.

4. An inhibitor of S. aureus SdrD protein attachment to a host cell comprising a recombinant SdrD A-region having the sequence shown in SEQ ID No. 3 or SEQ ID No. 4.

5. The inhibitor of claim 4, wherein said inhibitor has at least 95% homology to SEQ ID No. 3 or SEQ ID No. 4.

6. The inhibitor of claim 4, wherein said inhibitor has at least 90% or 80% homology to SEQ ID No. 3 or SEQ ID No. 4.

7. A pharmaceutical composition comprising the inhibitor of claim 4 a pharmaceutically acceptable carrier.

8. A method for treating or preventing a S. aureus infection of the lung in a subject in need thereof, comprising:
administering to the subject a pharmacologically effective amount of one or more of the small molecule inhibitors of claim 4.

9. The method of claim 8, further comprising:
administering one or more other therapeutic agents effective to treat the lung infection.

10. The method of claim 9, wherein the therapeutic agent(s) are administered concurrently or sequentially with the small molecule inhibitor.

11. The method of claim 8, wherein the lung infection is a nosocomial infection.

12. The method of claim 8, wherein the lung infection is pneumonia.

13. A method for inhibiting adherence of S. aureus bacteria to a lung cell, comprising:
contacting one or both of an S. aureus bacteria overexpessing SdrD protein or the lung cell with an amount of one or more of the small molecule inhibitors of claim 4 effective to interfere with attachment of S. aureus SdrD protein to its receptor on the lung cell thereby inhibiting adherence of the S. aureus bacteria.

14. A synthetic small molecule effective to bind to S. aureus SdrD protein or protein receptor.

15. An antibody directed against S. aureus SdrD protein.

16. The antibody of claim 15, wherein said SdrD protein is region A of the SdrD protein.

17. The antibody of claim 15, wherein said SdrD protein is the N2N3 region of the SdrD protein.

18. A method for treating or preventing an S. aureus-associated infection in a subject, comprising:
administering to the subject the antibody of claim 15.

19. The method of claim 18, wherein the S. aureus-associated infection is a nosocomial infection.

20. The method of claim 18, wherein the S. aureus-associated infection is pneumonia.

21. A method for treating or preventing an S. aureus-associated infection in a subject, comprising:
administering to the subject the antibody of claim 16.

22. A method for treating or preventing an S. aureus-associated infection in a subject, comprising:
administering to the subject the antibody of claim 17.

23. A small molecule inhibitor identified by the method of claim 1.

24. An immunogenic composition, comprising:
a protein from SdrD A-region having the sequence shown in SEQ ID No. 3 or SEQ ID No. 4.

25. The immunogenic composition of claim 24, wherein said protein has at least 95% homology to SEQ ID No. 3 or SEQ ID No. 4.

26. The immunogenic composition of claim 24, wherein said protein has at least 90% or 80% homology to SEQ ID No. 3 or SEQ ID No. 4.

27. The immunogenic composition of claim 24, further comprising an adjuvant.
